(19) United States
(12) Patent Application Publication
(10) Pub. No.: US 2007/0219429 A1
(43) Pub. Date: Sep. 20, 2007

(54) Privately Provisioned Interlocking Sub-Unit-Based Survival Supplies Provisioning Method

(76) Inventor: Barrett H. Moore, Winnetka, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/535,021

(22) Filed: Sep. 25, 2006

Related U.S. Application Data
(63) Continuation-in-part of application No. 11/384,037, filed on Mar. 17, 2006.
Application No. 11/384,037 is a continuation-in-part of application No. 11/394,350, filed on Mar. 30, 2006.
Application No. 11/394,350 is a continuation-in-part of application No. 11/279,333, filed on Apr. 11, 2006.
Application No. 11/279,333 is a continuation-in-part of application No. 11/379,929, filed on Apr. 24, 2006.
Application No. 11/379,929 is a continuation-in-part of application No. 11/381,247, filed on May 2, 2006.
Application No. 11/381,247 is a continuation-in-part of application No. 11/381,257, filed on May 2, 2006.
Application No. 11/381,257 is a continuation-in-part of application No. 11/381,265, filed on May 2, 2006.
Application No. 11/381,265 is a continuation-in-part of application No. 11/381,277, filed on May 2, 2006.
Application No. 11/381,277 is a continuation-in-part of application No. 11/383,022, filed on May 12, 2006.
Application No. 11/383,022 is a continuation-in-part of application No. 11/420,594, filed on May 26, 2006.
Application No. 11/420,594 is a continuation-in-part of application No. 11/421,694, filed on Jun. 1, 2006.
Application No. 11/421,694 is a continuation-in-part of application No. 11/423,594, filed on Jun. 12, 2006.
Application No. 11/423,594 is a continuation-in-part of application No. 11/425,043, filed on Jun. 19, 2006.
Application No. 11/425,043 is a continuation-in-part of application No. 11/426,231, filed on Jun. 23, 2006.
Application No. 11/426,231 is a continuation-in-part of application No. 11/456,472, filed on Jul. 10, 2006.
Application No. 11/456,472 is a continuation-in-part of application No. 11/461,605, filed on Aug. 1, 2006.
Application No. 11/461,605 is a continuation-in-part of application No. 11/461,624, filed on Aug. 1, 2006.
Application No. 11/461,624 is a continuation-in-part of application No. 11/462,795, filed on Aug. 7, 2006.
Application No. 11/462,795 is a continuation-in-part of application No. 11/462,845, filed on Aug. 7, 2006.
Application No. 11/462,845 is a continuation-in-part of application No. 11/464,751, filed on Aug. 15, 2006.
Application No. 11/464,751 is a continuation-in-part of application No. 11/464,764, filed on Aug. 15, 2006.
Application No. 11/464,764 is a continuation-in-part of application No. 11/464,775, filed on Aug. 15, 2006.
Application No. 11/464,775 is a continuation-in-part of application No. 11/464,788, filed on Aug. 15, 2006.
Application No. 11/464,788 is a continuation-in-part of application No. 11/464,799, filed on Aug. 15, 2006.
Application No. 11/464,799 is a continuation-in-part of application No. 11/465,063, filed on Aug. 16, 2006.
Application No. 11/465,063 is a continuation-in-part of application No. 11/466,727, filed on Aug. 23, 2006.
Application No. 11/466,727 is a continuation-in-part of application No. 11/466,953, filed on Aug. 24, 2006.
Application No. 11/466,953 is a continuation-in-part of application No. 11/470,156, filed on Sep. 5, 2006.
Application No. 11/470,156 is a continuation-in-part of application No. 11/531,651, filed on Sep. 13, 2006.
Application No. 11/531,651 is a continuation-in-part of application No. 11/532,461, filed on Sep. 15, 2006.
(60) Provisional application No. 60/823,806, filed on Aug. 29, 2006.

(51) Int. Cl. A61B 5/00

(52) U.S. Cl. 600/300

(57) ABSTRACT

A private civil security subscription mechanism serves to facilitate the provision of survival supplies for corresponding authorized beneficiaries. The survival supplies are segregated into physically interlocking sub-units. The physically interlocking sub-units of survival supplies can be fully or partially formed into a unit of supplies or can remain separate sub-units. The releasable nature of the interlocking sub-units allows the formation of the unit to be easily changed to respond to preferences and needs of the authorized beneficiary. If desired, an additional amount of supplies can be added to the unit if requested by the authorized beneficiary.

### RELATED APPLICATIONS

This application claims the benefit of the filing date of provisional application 60/823,806 entitled Private Civil Security Facilitation Method and Apparatus as was filed on Aug. 29, 2006.

This comprises a continuation-in-part of each of:

SUBSCRIPTION-BASED PRIVATE CIVIL SECURITY FACILITATION METHOD as filed on Mar. 17, 2006 and having application Ser. No. 11/384,037;

SUBSCRIPTION-BASED CATASTROPHE-TRIGGERED MEDICAL SERVICES FACILITATION METHOD as filed on Mar. 30, 2006 and having application Ser. No. 11/394,350;

PERSONAL PROFILE-BASED PRIVATE CIVIL SECURITY SUBSCRIPTION METHOD as filed on Apr. 11, 2006 and having application Ser. No. 11/279,333;

RADIATION SHELTER KIT APPARATUS AND METHOD as filed on Apr. 24, 2006 and having application Ser. No. 11/379,929;

FRACTIONALLY-POSSESSED UNDERGROUND SHELTER METHOD AND APPARATUS as filed on May 2, 2006 and having application Ser. No. 11/381,247;

SUBSCRIPTION-BASED CATASTROPHE-TRIGGERED TRANSPORT SERVICES FACILITATION METHOD AND APPARATUS as filed on May 2, 2006 and having application Ser. No. 11/381,257;

SUBSCRIPTION-BASED MULTI-PERSON EMERGENCY SHELTER METHOD as filed on May 2, 2006 and having application Ser. No. 11/381,265;

SUBSCRIPTION-BASED CATASTROPHE-TRIGGERED RESCUE SERVICES FACILITATION METHOD AND APPARATUS as filed on May 2, 2006 and having application Ser. No. 11/381,277;

DOCUMENT-BASED CIVILLY-CATASTROPHIC EVENT PERSONAL ACTION GUIDE FACILITATION METHOD as filed on May 12, 2006 and having application Ser. No. 11/383,022;

RESCUE CONTAINER METHOD AND APPARATUS as filed on May 26, 2006 and having application Ser. No. 11/420,594;

PURCHASE OPTION-BASED EMERGENCY SUPPLIES PROVISIONING METHOD as filed on Jun. 1, 2006 and having application Ser. No. 11/421,694;

SUBSCRIPTION-BASED PRE-PROVISIONED TOWABLE UNIT FACILITATION METHOD as filed on Jun. 12, 2006 and having application Ser. No. 11/423,594;

RADIATION-BLOCKING BLADDER APPARATUS AND METHOD as filed on Jun. 19, 2006 and having application Ser. No. 11/425,043; and

PRIVATE CIVIL DEFENSE-THEMED TELEVISION BROADCASTING METHOD as filed on Jun. 23, 2006 and having application Ser. No. 11/426,231;

EMERGENCY SUPPLIES PRE-POSITIONING AND ACCESS CONTROL METHOD as filed on Jul. 10, 2006 and having application Ser. No. 11/456,472;

PRIVATE CIVIL DEFENSE-THEMED BROADCASTING METHOD as filed on Aug. 1, 2006 and having application Ser. No. 11/461,605; and

METHOD OF PROVIDING VARIABLE SUBSCRIPTION-BASED ACCESS TO AN EMERGENCY SHELTER as filed on Aug. 1, 2006 and having application Ser. No. 11/461,624;

SUBSCRIPTION-BASED INTERMEDIATE SHORT-TERM EMERGENCY SHELTER METHOD as filed on Aug. 7, 2006 and having application Ser. No. 11/462,795;

SUBSCRIPTION-BASED CATASTROPHE-TRIGGERED RESCUE SERVICES FACILITATION METHOD USING WIRELESS LOCATION INFORMATION as filed on Aug. 7, 2006 and having application Ser. No. 11/462,845;

PRIVATELY PROVISIONED SURVIVAL SUPPLIES DELIVERY METHOD as filed on Aug. 15, 2006 and having application Ser. No. 11/464,751;

PRIVATELY PROVISIONED SURVIVAL SUPPLIES SUB-UNIT-BASED DELIVERY METHOD as filed on Aug. 15, 2006 and having application Ser. No. 11/464,764;

PRIVATELY PROVISIONED SURVIVAL SUPPLIES ACQUISITION METHOD as filed on Aug. 15, 2006 and having application Ser. No. 11/464,775;

PRIVATELY PROVISIONED SURVIVAL SUPPLIES CONTENT ACQUISITION METHOD as filed on Aug. 15, 2006 and having application Ser. No. 11/464,788;

METHOD TO PRIVATELY PROVISION SURVIVAL SUPPLIES THAT INCLUDE THIRD PARTY ITEMS as filed on Aug. 15, 2006 and having application Ser. No. 11/464,799;

WASTE DISPOSAL DEVICE as filed on Aug. 16, 2006 and having application Ser. No. 11/465,063;

SUBSCRIPTION-BASED PRIVATE CIVIL SECURITY RESOURCE CUSTOMIZATION METHOD as filed on Aug. 23, 2006 and having application Ser. No. 11/466,727;

PREMIUM-BASED PRIVATE CIVIL SECURITY POLICY METHODS as filed on Aug. 24, 2006 and having application Ser. No. 11/466,953;

SUBSCRIPTION-BASED MOBILE SHELTER METHOD as filed on Sep. 5, 2006 and having application Ser. No. 11/470,156;

METHOD OF PROVIDING A FLOATING LIFE-SUSTAINING FACILITY as filed on Sep. 13, 2006 and having application Ser. No. 11/531,651; and

PRIVATELY PROVISIONED SUB-UNIT-BASED SURVIVAL SUPPLIES PROVISIONING METHOD as filed on Sep. 15, 2006 and having application Ser. No. 11/532,461;

the contents of each of which are fully incorporated herein by this reference.

### TECHNICAL FIELD

This invention relates generally to the provision of survival supplies.

### BACKGROUND

Many citizens of the world have long passed the point when a ready availability of the basic necessities of life is satisfactory in and of itself. Today's consumer-oriented citizens demand, and often receive, an incredibly diverse and seemingly ever-growing cornucopia of consuming and experiential options. Such riches are typically based, in turn, upon a highly interdependent series of foundational infrastructure elements. Examples of the latter include, but are certainly not limited to:

transportation infrastructure such as roads, bridges, railways, and so forth that facilitate the inexpensive and rapid movement of sometimes perishable goods from source to consumer;

communications infrastructure such as telephones, television, radio, and the Internet that facilitate the inexpensive and rapid sharing of news, advice, information, and entertainment; and

the totality of civil services such as police services, fire fighting services, medical services, and so forth that facilitate a sufficient degree of order and predictability to, in turn, permit the complex series of inter-related actions that modern society requires in order to operate.

As powerful as the machinery of modern life appears, however, modern citizens are today perhaps more at risk of experiencing a serious disruption in their ability to prosper or even to survive en mass than is generally perceived. Providing the necessities of life in general requires a lot of things to all operate, more or less, correctly. To put it another way, a serious disruption to any significant element of civilized infrastructure can produce catastrophic results for a broad swath of a given civil community. Any number of natural and/or non-naturally-caused events can greatly disrupt society's infrastructure and ability to provide one or more life-sustaining resources such as water, nutrition, shelter, and the like.

This situation exists in large measure due to the just-in-time nature of modern inventory and control schemes and practices. As but one example, studies have shown that a typical modern urban grocery store has but a few days worth of inventory on hand at any given time. Without a virtually constant re-supply stream, shelves would quickly go bare. A significant disruption to supply chains, then, can lead to a rapid depletion of available stock. This, in turn, can lead to critical shortages of necessary emergency supplies at the very moment when such supplies are most needed. The unpredictability with respect to what supplies may become limited in this regard can comprise a particularly troubling component of this problem.

Many people believe and trust that their government (local, regional, and/or national) will provide for them in the event of such a civilly-catastrophic event. And, indeed, in the long view such is clearly a legitimate responsibility owed by any government to its citizens. That such is a consummation devoutly to be wished, however, does not necessarily make it so. Hurricane Katrina provided some insight into just how unprepared a series of tiered modern governmental entities may actually be to respond to even basic survival needs when a catastrophic event occurs.

Such insights, of course, are not particularly new. Civil preparedness shortcomings occasionally attract public attention and niche marketing opportunities exist with respect to provisioning the needs of so-called survivalists. Indeed, there are those who spend a considerable amount of their time and monetary resources attempting to ready themselves to personally survive a civilly-catastrophic event. Therein, however, lies something of a conundrum.

On the one hand, modern governments typically do little to proactively ensure the bulk provisioning (let alone the comfort) of their citizens in the face of many or most catastrophic events. On the other hand, attempting to take responsible actions to reasonably ensure one's own safety and security can become, in and of itself, nearly a full-time avocation and leave little time to actually enjoy the conveniences and opportunities of modern life. Such individual actions may even be frowned upon by the greater part of society which has grown accustomed to and falsely secure with existing efficient just-in-time delivery systems that provide the illusion of plenty while undercutting the perception of risk.

As a result, many (if not most) individuals and their families are largely bereft of the supplies that they will need should a civilly-catastrophic event befall them. This shortcoming tends to be relatively comprehensive; most people have neither a sufficient selection of survival supplies nor a sufficient quantity. For people who do have a store of supplies set aside against such an eventuality, it can be a considerable burden to maintain and ensure the freshness, vitality, and usability of those supplies. At the same time, the same civilly-catastrophic event that occasions their need for supplies will also likely disrupt relevant supply chains enough to cause a partial or complete shortage of supplies at their local merchants. The unfortunate net result is a relatively near term severe need for a variety of survival supplies that will often go unmet for lengthy periods of time.

### BRIEF DESCRIPTION OF THE DRAWINGS

The above needs are at least partially met through provision of the sub-units-based survival supplies provisioning method described in the following detailed description, particularly when studied in conjunction with the drawings, wherein:

FIG. 1 comprises a flow diagram as configured in accordance with various embodiments of the invention;

FIG. 2 comprises a schematic block diagram as configured in accordance with various embodiments of the invention;

FIG. 3 comprises a schematic block diagram as configured in accordance with various embodiments of the invention; and

FIG. 4 comprises a schematic block diagram as configured in accordance with various embodiments of the invention.

Skilled artisans will appreciate that elements in the figures are illustrated for simplicity and clarity. For example, common but well-understood elements that are useful or necessary in a commercially feasible embodiment are often not described in order to facilitate a less obstructed understanding of these various embodiments of the present invention. It will further be appreciated that certain actions and/or steps may be described or depicted in a particular order of occurrence while those skilled in the art will understand that such specificity with respect to sequence is not actually required. It will also be understood that the terms and expressions used herein have the ordinary meaning as is accorded to such terms and expressions with respect to their corresponding respective areas of inquiry and study except where specific meanings have otherwise been set forth herein.

### DETAILED DESCRIPTION

Generally speaking, pursuant to these various embodiments, a subscription-based approach serves to facilitate the procurement of survival supplies for corresponding authorized beneficiaries and to further facilitate the organization, management, and selection of such supplies. Various approaches are set forth with respect to the selection of the form and manner of providing, storing, and delivering the supplies through physically interlocking sub-units.

So configured, authorized beneficiaries of such consideration-based private civil security subscriptions will have concrete, predictable access to survival supplies upon the occurrence (and/or threat) of a civilly-catastrophic event. The selection and quantity of emergency survival items can be generally selected (and their maintenance governed) by experts and hence relieve the authorized beneficiary of responsibility in this regard.

These steps are readily facilitated without dependency upon governmental oversight, participation, or control. The particular supplies (and/or quantity of supplies) provided can vary with the needs and requirements of the authorized beneficiaries. Importantly, via these teachings, an individual can take important steps to bring a considerably improved measure of security into their lives, knowing that, should a civilly-catastrophic event indeed be visited upon them, they will have an extraordinary and reliable access to survival supplies.

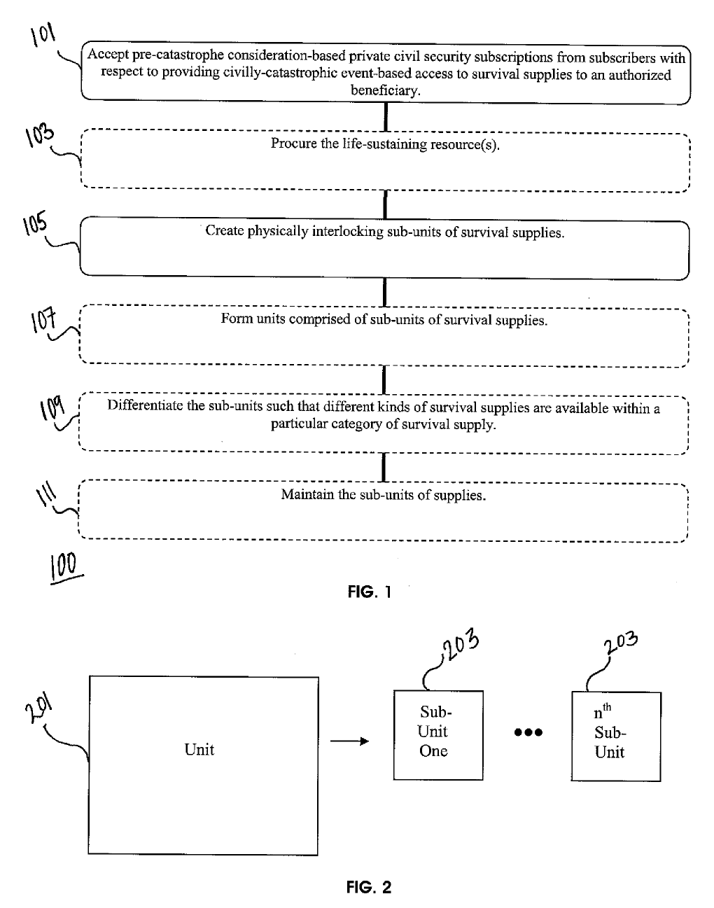

These and other benefits may become clearer upon making a thorough review and study of the following detailed description. Referring now to the drawings, and in particular to FIG. 1, an illustrative process 100 provides for accepting 101 pre-catastrophe consideration-based private civil security subscriptions from corresponding subscribers with respect to providing civilly-catastrophic event-based access to survival supplies. This right of access can pertain, if desired, to a predetermined period of time. For example, a given subscription can relate to providing access to the survival supplies for a one-year period of time for one or more authorized beneficiaries as correspond to that subscription.

By one approach, these subscriptions may be accepted by, for example, a for-profit business. By another approach a not-for-profit business (such as a membership-based-entity) may be the appropriate entity to offer and accept such subscriptions.

As noted, these teachings provide for a subscription-based approach. As used herein, the term “subscription” shall be understood to refer to and encompass a variety of legal mechanisms. Some relevant examples include, but these teachings are not limited to, subscription mechanisms such as:

time-limited rights of access (as where a subscription provides access rights for a specific period of time, such as one year, in exchange for a corresponding series of payments);

event-limited rights of access (as where a subscription provides access rights during the life of a given subscriber based upon an up-front payment in full and where those access rights terminate upon the death of the subscriber or where, for example, a company purchases a subscription for a key employee and those corresponding rights of access terminate when and if that key employee leaves the employment of that company);

inheritable rights of access (as may occur when the subscription, by its own terms and conditions, provides a right of access that extend past the death of a named subscription beneficiary and further provides for testate and/or intestate transfer to an heir);

rights of access predicated upon a series of periodic payments (as where a subscription provides access rights during, for example, predetermined periods of time on a periodic basis as where a subscriber offers month-by-month payments to gain corresponding month-by-month access rights);

rights of access predicated upon a one-time payment (as may occur when a subscriber makes a single payment to obtain a time-based or event-based duration of access rights or, if desired, when a single payment serves to acquire a one-time-only right of access or a perpetual right of access that may be retained, transferred, inherited, or the like);

ownership-based rights of access (as may occur when the subscription provides for ownership rights with respect to the survival supplies);

non-transferable rights of access (as may occur when the subscription, by its terms and conditions, prohibits transfer of the right of access to the survival supplies from a first named beneficiary to another);

transferable rights of access (as may occur when the subscription, by its terms and conditions, permits conditional or unconditional transfer of the right of access from a first named beneficiary to another);

membership-based rights of access (as may occur when the subscription, by its terms and conditions, establishes a membership interest with respect to the accorded right of access such as, for example, a club-based membership);

fractionally-based rights of access (as may occur when the subscription, by its terms and conditions, establishes a divided interest by and between multiple subscription beneficiaries with respect to a right to access the survival supplies);

non-ownership based rights of access (as may occur when the subscription, by its terms and conditions, establishes the aforementioned right of access via, for example, a lease, a rental, or borrowing construct);

option-based rights of access (as may occur when the subscription, by its terms and conditions, establishes a right for an authorized beneficiary to later obtain access to such resources upon, for example, paying an additional supplemental amount at that time).

If desired, a plurality of differentiated subscription opportunities can be offered in this regard. This plurality of differentiated subscription opportunities can correspond, for example, to providing access to differing selections and/or quantities of survival supplies. As but one very simple illustration in this regard, such subscription opportunities can differ from one another at least with respect to cost. This, in turn, provides subscriber choice with respect to selecting a particular subscription that best meets their specific needs and/or budget limitations.

These teachings also readily encompass the notion of a given subscriber providing such a subscription for an authorized beneficiary other than themselves. Such might occur, for example, when one family member procures such a subscription for one or more other family members. Another example would be for a company to subscribe on behalf of named key employees, family members of such key employees, and so forth. Other examples no doubt exist.

As noted, these subscriptions relate to providing access to survival supplies in the event of a civilly-catastrophic event. Such access may be predicated, if desired, upon a requirement that the civilly-catastrophic event be one that persists in substantial form for more than a predetermined period of time (such as one hour, one day, one week, and so forth) or that causes at least a predetermined amount or degree of infrastructure impairment or other measurable impact of choice. In addition, or in lieu thereof, such access may be predicated, if desired, upon a requirement of a particular level of objectivity or subjectively ascertained likelihood that a particular category or kind of civilly-catastrophic event will occur within a particular period of time.

As used herein, “civilly-catastrophic event” will be understood to refer to an event that substantially and materially disrupts a society's local, regional, and/or national infrastructure and ability to provide in ordinary course for at least one life-sustaining resource. Such a civilly-catastrophic event can include both a precipitating event (which may occur over a relatively compressed period of time or which may draw out over an extended period of time) as well as the resultant aftermath of consequences wherein the precipitating event and/or the resultant aftermath include both the cause of the infrastructure interruption as well as the continuation (or worsening) of that interruption.

A civilly-catastrophic event can be occasioned by any of a wide variety of natural and/or non-naturally-caused disasters. Examples of natural disasters that are potentially capable of initiating a civilly-catastrophic event include, but are not limited to, extreme weather-related events (such as hurricanes, tsunamis, extreme droughts, widespread or unfortunately-targeted tornadoes, extreme hail or rain, and the like, flooding, and so forth), extreme geological events (such as earthquakes, volcanic activity, and so forth), extreme space-based events (such as collisions with comets, large asteroids, and so forth, extreme solar flares, and the like), extreme environmental events (such as widespread uncontrolled fire or the like), and global or regional pandemics, to note but a few.

Examples of non-naturally-caused disasters capable of initiating a civilly-catastrophic event include both unintended events as well as intentional acts of aggression such as war, terrorism, madness, or the like. Examples of non-naturally-caused disasters capable of such potential scale include, but are not limited to, nuclear-related events (including uncontrolled fission or fusion releases, radiation exposure, and so forth), acts of war, the release of deadly or otherwise disruptive biological or chemical agents or creations, and so forth.

This process 100 also provides for acquiring or procuring 103 the corresponding survival supplies for the authorized beneficiaries. Such procurement can be achieved through any of a variety of means. By one approach the items may be acquired on the open market. By another approach the items may be purchased or otherwise acquired from third parties via private negotiations. By yet another approach the entity that provides and accepts these subscriptions may itself create (through manufacturing, farming, or the like) the items of interest. In some cases, the acquired item may comprise a staple of ordinary commerce. In other cases, the acquired item may be unique and/or proprietary to the acquiring/storing entity.

This process 100 then provides for creating 105 physically interlocking sub-units of survival supplies. The interlocking sub-units include an engagement mechanism or locking structure that can secure the sub-units relative to one another. In a preferred form, the interlocking sub-units are discrete components of survival supplies that can be releasably fixed together.

In one form, creating 105 interlocking sub-units may include segregating supplies and packaging the supplies into a container that includes the engagement mechanism or locking structure. The container may be a bin, a box, a case, a drum, a frame, a chamber, a bucket, a basket, a bag, a trunk, a carton, a chest, a coffer, a drawer, a cabinet, a pot, a pan, a jar, and/or a barrel, to note a few. The container may be any of a variety of shapes and sizes. The container may be at least one of: molded plastic (such as polypropylene, polyethylene, acrylonitrile butadiene styrene, polycarbonate, polyvinyl chloride, polyethylene terephthalate, high impact polystyrene, and so forth), corrugated plastic, hard shell plastic, Teflon, corrugated cardboard, wood, steel, various carbon-based materials, aluminum, composite material, and glass, to note only a few. In another form, the supplies themselves include the engagement mechanism or locking structure and can directly be locked to other sub-units. For example, the supplies themselves are not positioned or packaged within a container but already comprise discrete supplies that can be fixedly attached to other sub-units of supplies. Such a sub-unit may be advantageous with larger survival supplies having a durable outer surface such that a locking structure can be fixedly attached thereto or can be incorporated into the design of the supplies themselves. In addition, removing any extraneous packaging surrounding such supplies decreases the weight of the sub-unit.

If a container is used to segregate the supplies into sub-units, the container employed typically includes a base with a number of substantially vertical sidewalls and a lid that is at least partially removable to thereby access the contents of the container. In one preferred form, there are four substantially vertical sidewalls. The lid can engage all or at least a substantial portion of the sidewalls. The engagement between the lid and the remainder of the container can include: a hinge, a magnet (such as a permanent magnet or an electromagnet), a snap fit, a clip, a press-fit structure, a hooks-and-loops fastener, a hook, a latch, a clamp, a formed joint, a bolt, a cable, a keyed coupler and interlocking form, to note a few. The lid may be permanently attached, such as by a hinge, or removably attached, such that the lid may be completely removed from the container. Whether or not the lid can be completely removed, the lid includes structure allowing it to be secured to the container such that the lid remains firmly attached thereto during shipping and storage. In another form, the base of one of the sub-units is configured to selectively serve as the lid for another sub-unit. For example, if the sub-units are stacked atop one another, the bottom surface of one base directly faces the supplies in the sub-unit underneath. This reduces the amount of material used and thereby may decrease the total weight of the unit. However, if the base of one sub-unit selectively provides the lid for another sub-unit, the sub-unit atop the other sub-units may still be provided a separate lid.

Whether the supplies are packaged into a container or can be directly attached to the other sub-units, the interlocking sub-units utilize a locking structure to secure the sub-units together. The locking structure may include at least one of: a magnet (such as a permanent magnet or an electromagnet, which could be powered by a generator integrated into the unit), a snap fit, a clip, a coupling structure, a press-fit structure, a hooks-and-loops fastener, a hook, a latch, a threaded structure, a clamp, a formed joint, a cable, and a keyed coupler, to note a few. In one form, a plurality of these locking structures will be incorporated into one sub-unit locking structure. As discussed below, when forming the sub-units of survival supplies, the sub-units can be horizontally, vertically, and/or diagonally secured to one another. Therefore, the locking structure preferably can be mounted, attached, or incorporated into nearly every exterior surface of the sub-unit.

The container may include a number of indentations and projections for engaging or mating with the projections and indentations of other sub-units. This can comprise the locking structure or can comprise a supplemental additional locking structure. The container may also include ribbed reinforcements to strengthen the container. In addition, the container may include a number of handles for grasping or other surfaces to facilitate manipulation or movement of the container by corresponding apparatus (such as a forklift or a crane). Such handles may also mate or engage with structure on other sub-units.

In one embodiment, each of the physically interlocking sub-units is dedicated to like supplies. This can comprise, for example, creating a plurality of discrete sub-units dedicated to particular discrete categories of like survival supplies. Having sub-units dedicated to a particular category of like resources organizes the sub-units and may be a useful tool when delivering the survival supplies to the authorized beneficiaries, in response to a civilly-catastrophic event. The discrete categories of sub-units might include, but are not limited to:
a nutritional and hydration supplies category (which may include fresh foods, dehydrated goods, nitrogen-packed foods, canned foods, freeze-dried foods, frozen foods, refrigerated foods, raw foods, processed foods, preserved foods, potable water, bottled water, vitamin supplements, water purification apparatus, water purification chemicals, sports drinks, energy drinks, and so forth):
a shelter supplies category (which may include lumber, nails, shingles, concrete, tools, tarps, tents, and so forth);
a sundries category (which may include personal hygiene items, toiletries for different ages and genders, batteries, flash lights, matches, lighters, thread, needles, and other sewing supplies, facial and body care products, and so forth);
a tools category (which may include hunting and gathering tools, repair and maintenance tools, such as a hammer, nails, screw driver, screws, saw, tape measure, glue, tape, level, and so forth);
a medical and pharmaceutical supplies category (which may include first aid supplies, healthcare supplies, life support supplies, medicines, vaccines, specific illness related supplies, specific disease related supplies, long term care supplies, consumable medical supplies, non-consumable medical supplies, treatment equipment, stabilization equipment, long-term care equipment, diagnostic equipment, and so forth);
a clothing category (which may include adult, adolescent, child, infant, male, female, transgender, unisex or general clothing, suitable for various climate conditions, weather conditions, locations, likely threats, seasons, and so forth);
a communication category (which may include one-way communication facilities, two-way personal communication facilities, a wireless communication device, walkie-talkies, and so forth);
an entertainment category (which may include media supplies, games, games, reading material, sports equipment, and so forth);
a camping category (which may include a tent, a tarp, tools, lighting, power generation equipment, communication equipment, entertainment supplies, transportation vehicles, fire starting supplies, navigation equipment and aids, survival instructions, weapons, pack, load bearing equipment, and so forth);
a nutritional supplements and food preparation supplies category (which may include vitamin and mineral supplements, cookware, bakeware, a heat source, such as a stove, oven, gas-filled or charcoal grill, fire starting aids, a can opener, eating utensils, cutlery, dinnerware, glassware, and so forth);
a lighting category (which may include power generation equipment, lights, lamps, lanterns, fuels, flash lights, fire starter, and so forth);
a power generation category (which may include electrical energy sources such as alternating current power sources, direct current power sources, fuel-consumptive power sources, renewable fuel source power sources, solar cells, generators, and so forth);
a transportation apparatus and supplies category (which may include a terrestrial vehicle, a water-borne vehicle, an air-borne vehicle, a hovercraft, and so forth);
a self-defense supplies category (which may include breathable air supplies, gas masks, filtered masks, particle masks, smoke hoods, face masks, head gear such as protective hats or helmets, firearms, ammunition, armor, debilitating sprays, other weapons such as projectile-based weaponry, blunt or sharp instruments, electromagnetic pulse protection or recovery supplies, and so forth);
a nuclear, biological, and chemical threat-abatement supplies category (which may include a gas mask, a filtered mask, breathable air supplies, sanitation supplies, radiation exposure related supplies, such as a radiation resistant suit, potassium iodide, wearable dosimeter, and so forth, chemical exposure related supplies, biological threat exposure related supplies, signal device, body armor, limb protection supplies, tools, bio-hazard suit, radiation suit, helmet, visor, ear protection, eye protection, and so forth);
a twenty-four hour supply kit category (which may include supplies sufficient for surviving a given consecutive twenty-four hour period of time such as: a basic communication device such as a prepaid cellular telephone, walkie talkies, a signal mirror, and so forth; nutritional supplies such as food, vitamins and other dietary supplements; shelter supplies, such as a tent, sanitary facilities and supplies, tarps, and so forth; entertainment supplies, such as games, reading materials, audio and video platforms, and content; sundries, and so forth);
a seventy-two hour supply kit category (which may include supplies sufficient for surviving a given consecutive seventy-two hour period of time: a basic communication device, such as a prepaid cellular telephone, walkie talkies, a signal mirror, and so forth; nutritional supplies such as food, vitamins and other dietary supplements; shelter and camping supplies, such as a tent, sanitary facilities and supplies, tarps, and so forth; entertainment supplies, such as games, reading materials, audio and video platforms, and content; sundries, such as personal hygiene items, laundry products, and so forth);
a seven day supply kit category (which may include supplies sufficient for surviving a given consecutive seven-day period of time: a communication device, such as a prepaid cellular telephone, walkie talkies, a signal mirror, antenna tower, cellular telephone signal amplifier, and so forth; nutritional supplies such as food, vitamins and other dietary supplements; shelter and camping supplies, such as a tent, sanitary facilities and supplies, tarps, food preparation tools, cutting tools, inflatable and/or folding furniture, bedding supplies, such as sleeping bags, and so forth; entertainment supplies, such as games, reading materials, audio and video platforms, and content; sundries; tools; lighting supplies; clothing supplies; medical supplies, such as first aid supplies, diagnostic equipment, medicines of various kinds; transportation supplies such as a collapsible bike, inflatable boat, and so forth; power generation supplies, such as batteries, generators, solar cells and so forth; threat abatements and self-defense supplies, and so forth);
a ten day supply kit category (which may include supplies sufficient for surviving a given consecutive ten-day period of time such as: a communication device, such as a prepaid cellular telephone, walkie talkies, a signal mirror, an antenna tower, a cellular telephone signal amplifier, and so forth; nutritional supplies such as food, vitamins and other dietary supplements; shelter and camping supplies, such as a tent, sanitary facilities and supplies, tarps, food preparation tools, cutting tools, inflatable and/or folding furniture, bedding supplies, such as sleeping bags, and so forth; entertainment supplies, such as games, reading materials, audio and video platforms, and content; sundries; tools;
lighting supplies; clothing supplies; medical supplies, such as first aid supplies, diagnostic equipment, medicines of various kinds; transportation supplies such as a collapsible bike, inflatable boat, and so forth; power generation supplies, such as batteries, generators, solar cells and so forth; threat abatements and self-defense supplies, and so forth);
a fourteen day supply kit category (which may include supplies sufficient for surviving a given consecutive fourteen-day period of time such as: a communication device, such as a prepaid cellular telephone, walkie talkies, a signal mirror, an antenna tower, a cellular telephone signal amplifier, and so forth; nutritional supplies such as food, vitamins and other dietary supplements; shelter and camping supplies, such as a tent, sanitary facilities and supplies, tarps, food preparation tools, cutting tools, inflatable and/or folding furniture, bedding supplies, such as sleeping bags, and so forth; entertainment supplies, such as games, reading materials, audio and video platforms, and content; sundries; tools; lighting supplies; clothing supplies; medical supplies, such as first aid supplies, diagnostic equipment, medicines of various kinds; transportation supplies such as a collapsible bike, inflatable boat, and so forth; power generation supplies, such as batteries, generators, solar cells and so forth; threat abatements and self-defense supplies, and so forth);
a thirty day supply kit category (which may include supplies sufficient for surviving a given consecutive thirty-day period of time such as: a communication device, such as a prepaid cellular telephone, walkie talkies, a signal mirror, an antenna tower, a cellular telephone signal amplifier, and so forth; nutritional supplies such as food, vitamins and other dietary supplements; shelter and camping supplies, such as a tent, sanitary facilities and supplies, tarps, food preparation tools, cutting tools, inflatable and/or folding furniture, bedding supplies, such as sleeping bags, and so forth; entertainment supplies, such as games, reading materials, audio and video platforms, and content; sundries; tools; lighting supplies; clothing supplies; medical supplies, such as first aid supplies, diagnostic equipment, medicines of various kinds; transportation supplies such as a collapsible bike, inflatable boat, and so forth; power generation supplies, such as batteries, generators, solar cells and so forth; threat abatements and self-defense supplies, and so forth);
   to note a few. For example, all of the food, potable water, and other nutritional and hydration supplies can be segregated and grouped together as a shared or common sub-unit. The short term supply kits detailed above include one, three, seven, ten, fourteen, and thirty day denominations; however, an authorized beneficiary may request a number of short term supply kits suitable for varying periods of time. For example, a beneficiary may request a one day supply kit and a one hundred day supply kit.

In addition, the interlocking sub-units may be further compartmentalized. For example, the nutrition and hydration sub-unit may be separately grouped such that within the nutritional and hydration category there are separate sub-categories for hydration and nutrition. Such further compartmentalization may encourage or facilitate proper stocking and rotation of inventory, packing, packaging, storage, pre-positioning, and delivery. Like the interlocking sub-units themselves, the compartments of a sub-unit may be stored separately. For example, within the nutritional and hydration supplies sub-unit, the nutrition compartment may need to be frozen whereas the hydration compartment may require room-temperature storage. The interlocking sub-units may be compartmentalized based upon a number of factors, such as, for example, when the resources must be rotated or otherwise updated to ensure quality. Such rotational maintenance may be expedited due to the structure of the unit through sub-unit and compartmentalization as discussed below. The compartments of the sub-unit may also be discrete interlocking components.

In one form, creating 105 the interlocking sub-units may include segregating the supplies, such as by partitioning off the supplies of one authorized beneficiary from a larger aggregation of collective supplies. Further, such segregation of supplies may comprise encapsulating, bundling, or otherwise restraining the removed supplies. For example, segregating can include removing a carton or small box of supplies from a larger bin or larger pallet of aggregated supplies. In another form, the segregation can occur by gathering different supplies, possibly procured from different sources, into a sub-unit. Segregating supplies occurs, for example, when a flashlight, a tent, a lantern, fire starter, walkie-talkies, and matches are acquired and then are combined to create the camping supplies sub-unit. In yet another form, creating 105 the sub-unit may comprise both partitioning off a portion of supplies and combining supplies from different sources.

By creating 105 interlocking sub-units of survival supplies, the supplies are provided with an increased level of organization. Upon receipt of the supplies, the authorized beneficiary can quickly find items without having to organize or inventory the items received. The sub-unit organization makes the supplies easier to locate and access when they are needed by the authorized beneficiary. Upon occurrence of a civilly-catastrophic event, many individuals may experience some confusion, anxiety, and fear among other emotions. It is during these emotionally charged moments that authorized beneficiaries will likely need access to a number of the supplies. Therefore, storing the supplies in a coordinated and systematic manner can increase the likelihood that an authorized beneficiary will be able to find particular supplies when needed most.

To assist the authorized beneficiary in finding particular supplies, a supply list providing the authorized beneficiary with sub-unit and compartment information can accompany the resources. The sub-units may be stored or packaged in a manner to distinguish the sub-units from one another. Further, such identifying characteristics may be included in the supply list. For example, the sub-units or the sub-unit packaging may be color coded or affixed with printed graphics, symbols, letters, number, or other indicia. In another embodiment, any item requiring prompt access can be provided with an alarm or tracking device that can indicate the item's location. It is also contemplated that particular interlocking sub-units themselves can be provided with indicators, alarms, or tracking devices for quick location. In yet another form, the sub-unit containers or a portion thereof are transparent, such that the supplies stored therein can be seen and identified without opening or unlocking the interlocking sub-unit. By one example, portions of the container comprises ventilation openings allowing for visibility and/or air flow. In another form, the authorized beneficiary may receive a map or diagram such that particular sub-units can be more easily located. Such a map would provide the location of a particular sub-unit relative to the other sub-units of the unit.

Creating 105 interlocking sub-units can provide segregation between supplies that have a high potential for reactivity, flammability, and contamination from other supplies. In addition, certain storage requirements regarding such factors as air pressure, air quality, humidity, temperature, light sensitivity, and moisture levels can be more easily accommodated by having separately stored supplies.

In addition, storing supplies in sub-units allows the storage containers to be customized to the supplies stored therein. Depending on the category of supplies, the container may need to be: double walled, reinforced, heat resistant, non-conductive, insulated, lightweight, durable, corrosion resistant, or refrigerated, to name a few. For example, while certain nourishment supplies may require storage in heat resistant packaging the clothing supplies do not require such accommodations and therefore may be stored more cost effectively in other packaging. Therefore, the locking structure can preferably be incorporated into containers comprised of different materials.

The interlocking sub-units may be stored separately at different locations or under different conditions. Storing the sub-units at separate locations minimizes the risk associated with the loss of resources. For example, supplies can be lost due to weather damage, natural disasters, looting, animal contamination, spoiling, and so forth. Sub-units may be stored in different geographic locations that may have varying climate or population conditions and may be exposed to different likely threats. In addition to reducing the risk of resource loss, some resources may be better preserved being stored at subterraneous locations. Thus, for supplies that can be stored for long periods of time, subterranean storage is possible since those supplies can be segregated from the perishable items that require more frequent access. When the authorized beneficiary may require access to the supplies might affect where the supplies are stored. Since the sub-units may also be stored in varying distances from the authorized beneficiary, it may be helpful to store the short term supplies within relative proximity to the authorized beneficiary.

This process 100 also provides for forming 107 the interlocking sub-units into a unit of survival supplies. As shown in FIG. 2, such a unit 201 is comprised of a plurality of smaller interlocking sub-units 203. This can comprise assembling or locking together all of the interlocking sub-units for one authorized beneficiary. Alternatively, forming 107 the unit can comprise assembling or locking together portions or a few, but not all, of the sub-units for one authorized beneficiary.

Assembling the entire unit is beneficial for quick dispatch of the supplies. However, assembling only a portion of the total sub-units allows for separate storage of the sub-units. This can comprise having a partially disassembled unit or sub-unit to accommodate different storage locations or different storage conditions, such as air quality, and temperature. For example, some supplies may be suitable for storage at room temperature, while other supplies may require or benefit from frozen storage. By way of another example, an authorized beneficiary may need convenient access to a short term supply sub-unit (such as a twenty-four hour, seventy-two hour, seven day, ten day, fourteen day, or thirty day short term supply sub-unit) and therefore, those sub-units may be stored at the authorized beneficiary's home or at a storage facility relatively proximal to the authorized beneficiary's home or principal location. The usefulness of the supplies can be affected by how quickly the authorized beneficiary can receive her supplies and therefore proximity of the supplies can be of significant importance. It may be beneficial to store certain sub-units at different locations relatively distal from one another. Some supplies may not be able to be stored together due to a risk of reactivity, or potential for contamination. Certain supplies will likely require more frequent access than others and storing a number of these supplies together can prevent the maintenance staff from disassembling the entire unit to change or rotate a few supplies. For example, if the medical and pharmaceutical supplies are frequently updated to track an authorized beneficiary's changing medical condition, then access to that sub-unit may be required more frequently than other sub-units. Thus, forming 107 the units can encompass all or most of the sub-units or just a few of the sub-units with other sub-units to be added at a later point in time.

While the interlocking sub-units can be stored separately from one another, the locking structure allows the separate sub-units to be quickly adapted into the unit. In sum, the locking structure allows for the quick removal, addition, or exchange of sub-units in the unit. If portions of the units that are stored separately from other interlocking sub-units, the unit can be quickly prepared for delivery to the authorized beneficiary because the locking structure provides a manner of forming the unit that is incorporated into the storage of the supplies and the supplies themselves.

The method of assembling and forming 107 the unit may depend on the sub-unit and the locking structure. In one preferred embodiment, the locking structure is releasable such that the units can be formed into one arrangement and then reformed into a different arrangement or with different sub-units if desired. For example, the supplies of the sub-unit may be stored in bins with formed joints and tabs having hooks-and-loops fasteners. Thus, forming the unit may entail mating the respective portions of the formed joints and securing the hooks-and-loops fastener tabs of at least two of the individual interlocking sub-units. If the sub-units are coded, such as by color or graphics, forming 107 the unit may require examining the indicia to ensure the inclusion of particular coded sub-units.

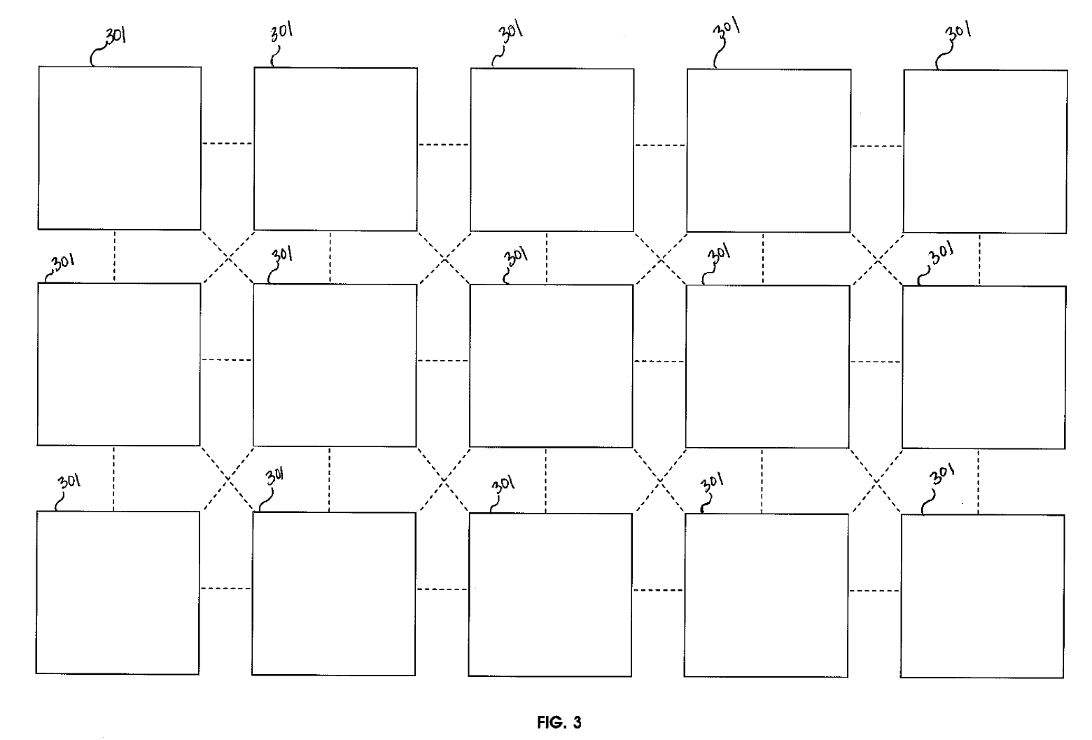

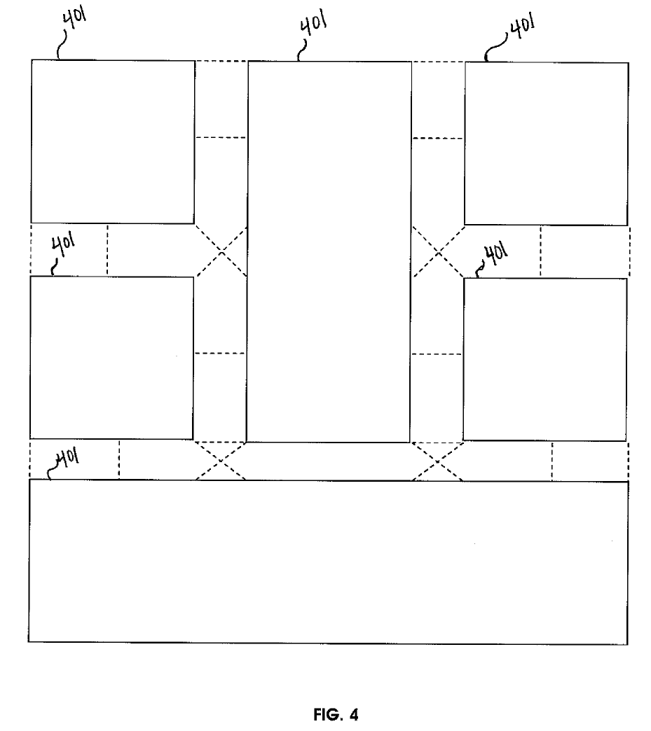

The units formed can include different sizes, shapes, and numbers of sub-units. For example, it may be beneficial for an authorized beneficiary to store the sub-units in a space that is long, with minimal clearance. Therefore, the sub-units may not be stacked but instead are stored level with one another. In FIG. 3, subunits 301 are schematically shown in one layer. The dashed lines shown illustrate that the locking structure can secure the sub-units relative to one another in different directions. Horizontal, vertical, and diagonal securement mechanism may be employed in varying combinations to lock the sub-units together. FIG. 4 illustrates a number of differently sized and shaped sub-units 401 forming a unit. Differently sized sub-units may better accommodate unusually shaped or sized supplies. It is contemplated that additional sub-units may be included stacked atop the sub-units shown.

In one embodiment, a basic or standard sub-unit is available within each supply category. The standard sub-units can be formed together to form a universal unit. This universal unit preferably includes the staple items that an authorized beneficiary will require to sustain themselves for a certain period of time. The universal unit is preferably a largely unisex, uni-age, one-size-fits-all selection of supplies. Utilizing universal units allows for some convenience with respect to managing the resources. The unit enjoys a certain level of fungibility such that the unit received by one authorized beneficiary is nearly identical to that received by another. As described below, the sub-units may be differentiated and a sub-unit in a particular category can be exchanged for another sub-unit in that category. If the authorized beneficiary elects to differentiate a particular sub-unit, the function of the sub-unit remains the same and the unit retains a level of functional fungibility. In yet another form, the universal unit comprised of standard sub-units can be supplemented by add-ons, such that an authorized beneficiary can request additions while retaining the standard sub-unit supplies. By one approach, a number of additional interlocking sub-units will include the additional resources requested by the authorized beneficiary and then those additional sub-units can be added to the unit by locking the additional sub-units to the standard interlocking sub-units. By having a universal unit with added features, the authorized beneficiary has a partially customized unit without having to pay for an entirely customized selection.

Standardization of at least a portion of the supplies may also increase the ability to quickly deliver supplies to an authorized beneficiary that has traveled away from her home or principal location. If a civilly-catastrophic event occurs when the authorized beneficiary is relatively distal from the beneficiary's unit, the standard or universal sub-units may be supplied from a stock of resources more proximal to the beneficiary. Further, if the authorized beneficiary has added a few additional customized sub-units only those sub-units will need to be shipped to the location of the authorized beneficiary. Thus, having a standard or universal unit with a few additional customized interlocking sub-units may provide the authorized beneficiary with a level of increased mobility.

In another form, the differentiated sub-units can be combined in various combinations to create a unit that is more personal to the authorized beneficiary receiving the unit than the universal unit. In one form, only a few of the differentiated sub-units will be exchanged for standard sub-units such that the unit is comprised of both standard and differentiated sub-units. In another form, the unit may be comprised entirely of differentiated sub-units such that the unit has a high level of customization. Thus, the personal unit provides the authorized beneficiary, who has upgraded particular categories of supplies to one of the differentiated sub-units, a partially customized unit without having to pay for an entirely customized selection of supplies.

Incorporating interlocking sub-units allows for the unit to be more quickly updated to the authorized beneficiary's changing preferences and requirements. The locking structure is preferably releasably interlocking such that the sub-units can be locked, unlocked, and then relocked in different configurations if so desired. This also can aid in the maintenance of the supplies as discussed below.

The process 100 also provides for differentiating 109 the sub-units of survival supplies within a category of supplies. The differentiated sub-units correspond to the same particular discrete category of survival supplies, but contain different kinds or types of survival supplies within one category of supplies. The difference in supplies can be quite small or can become quite significant. For example, within the clothing sub-unit category, differentiated sub-units may be available that contain adult female or adult male clothing. Further, the standard sub-unit included in the universal unit may contain unisex clothing that can be used for a broad range of ages and sizes. By way of another example, the tools category may be differentiated by the number of separate tools included in the sub-unit: one sub-unit may include a number of handheld power tools while another sub-unit may include a single table-top or bench tool that can accomplish the same tasks. It is contemplated that the tools provided in the sub-unit are generally sufficient to accomplish virtually any tasks, such as constructing shelter or fixing vehicles. Thus, while each of the tools sub-units may include sufficient equipment to accomplish a variety of tasks, the tools provided to complete such tasks can vary from one sub-unit to another.

Providing for differentiation of the sub-units allows the unit to be tailored to the preferences, requirements, and needs of the authorized beneficiary in addition to the conditions and requirements of potential civil-catastrophes. Differentiated sub-units also provide a level of customization without having to construct an entire unit by deciding on each detail and starting from scratch. In one form, the differentiated sub-units are predetermined such that the authorized beneficiary has a number of choices within each supply category. Differentiation of the sub-units included in a unit creates a semi-customized unit because an authorized beneficiary can choose her sub-units from a selection of available differentiated sub-units.

In one embodiment, the differentiated sub-units are functionally fungible with respect to one another. The sub-units in such cases can be exchanged for one another. Having sub-units with a level of interchangeability allows the sub-units to be more easily substituted for one another when formed into a unit and as stated earlier releasably interlocking sub-units can increase the interchangeability. In addition, increased fungibility can be particularly beneficial with respect to effecting the proper stocking and rotation of inventory, packing, packaging, storage, pre-positioning, and delivery of such survival supplies.

By one approach, it is expected that each category of sub-unit will have at least one differentiated sub-unit. These sub-units can be differentiated based on a number of factors that might include, but are not limited to:
age of the authorized beneficiary;
experience of the authorized beneficiary (such as whether the authorized beneficiary will have the skill level to employ the use of certain supplies, and so forth);
interests of the authorized beneficiary;
gender of the authorized beneficiary;
location (such as the principal location of the authorized beneficiary, the storage location of a plurality of the survival supplies and the most likely threat at the principal location of the authorized beneficiary, and so forth);
likelihood of use (such as whether the authorized beneficiary will be able to use the supplies and the probability of whether certain situations will materialize, and so forth);
cost;
brand;
packaging;
size;
portability (such as size and weight considerations, whether the supplies include wheels, runners, handles, and so forth);
durability;
accessibility;
availability (such as whether the supplies are easy or difficult to procure, and so forth);
ease of use;
training requirements;
season and seasonal requirements;
color;
smell;
noise;
taste;
touch;
compatibility;
interoperability;
threat assessment;
additional supplies provided;
medical conditions (including whether an authorized beneficiary suffers from a particular medical condition that requires treatment or whether an individual's general medical condition requires preventative measures to avoid developing certain medical problems, and so forth);
information included;
shape;
composition;
efficacy;
personal preference;
type of material;
number of individuals that can utilize the supplies;
number of individuals accommodated;
weather requirements;
power production capacity;
fuel requirements;
weight;
the degree to which the supplies can be concealed;
fire power;
caliber;
rate of fire;
threat mitigation;
viability;
length of protection;
functionality;
nutritional value;
effectiveness;
   to note but a few examples. By one approach, for example, differentiation based on gender may comprise having male specific clothing in one clothing sub-unit and female-specific clothing in another clothing sub-unit. Along these same lines, it would also be possible for the sub-units to comprise supplies that are at least primarily directed to a particular age range. Similarly, it is also contemplated that the sub-units will be directed to different sizes of beneficiary. For example, the clothing may be provided in sizes ranging from petite dimensions to very large dimensions.

The process 100 also optionally provides for maintaining 111 the interlocking sub-units of survival supplies pending a need to permit subscription-based access to the supplies. The specifics of such maintenance will of course vary with respect to the nature of the resource or resources being maintained and the preferences of the authorized beneficiary and/or subscriber. As suggested above, the supplies may be stored in unit form or in a partial unit. Grouping or segregating the supplies into sub-units may significantly impact the maintenance process. Maintaining the supplies may comprise ensuring the utility of the stored supplies. The supplies may need to be updated on a periodic basis to ensure, for example, freshness, usability, and efficacy of the supplies. In one form, this can comprise monitoring the usability of perishable items and replacing such items on a corresponding schedule. As another simple illustration, this step can comprise holding certain items in deep refrigeration or in other special storage conditions as may suit the proper maintenance of such items.

Such maintenance can also optionally comprise making adjustments to such supplies to reflect dynamically changing circumstances as occur during the consideration-based private civil security subscription period. Advancements in technology will often produce more efficient and effective resources, such that the previously stored resources will need to be substituted. As one illustration, a new item may become available that is particularly useful in dealing with or otherwise surmounting some condition that may likely arise upon the occurrence of a particular kind of civilly-catastrophic event. In such a case, maintaining such supplies can readily accommodate updating the acquired and stored items to include a supply of this new item.

Accordingly, such maintenance can readily comprise one or more of removing a particular one of the items (as when a better substitute becomes available, when the item itself is shown to be less effective for its intended purpose than was originally thought, when the authorized beneficiary has chosen to receive another item, and so forth), adding additional ones of the stored supply (as when it becomes subsequently understood that more of a particular item is desired to achieve a particular survival related goal or purpose, or an authorized beneficiary has changed preference), adding at least one new supply that is not already stored (as in the example provided above) and so forth. Whether removing, adding, or exchanging stored items, the releasable interlocking sub-units allow for easier access to the supplies since the sub-units can be conveniently locked and unlocked.

As suggested, maintenance of the survival supplies and resources is assisted by the segregation of the survival supplies into sub-units and compartments. These divisions can facilitate better, more efficient and effective, organization, management, and selection of supplies. Supplies that require frequent maintenance and need to be more easily accessible can be stored in a more convenient location. If desired, the sub-units requiring such frequent access can be stored separate from other sub-units of the unit or can be stored in a relatively accessible portion of the unit. For example, if the medical and pharmaceutical sub-unit requires maintenance on a more frequent basis than the most of the other sub-units, the medical and pharmaceutical sub-unit can be stored separately from the other sub-units. Further, a plurality of the medical and pharmaceutical sub-units can be stored together such that when maintenance (such as removing, exchanging, or adding an item) must occur, all of the sub-units requiring such maintenance can be efficiently updated.

It will be appreciated that these teachings provide for a highly flexible yet powerfully effective way by which a modern citizen can greatly improve their likelihood of surviving a civilly-catastrophic event. These teachings are sufficiently flexible so as to accommodate the needs and desires of a wide-ranging set of potential beneficiaries while nevertheless still tending to ensure adequate access to the basic necessities of life. Further, the teachings provide a manner to accommodate the individual beneficiary while creating a viable manner to implement such a large-scale provision of survival supplies. Though training and some related activity may be provided and encouraged, in general the beneficiary receives those benefits without being required to make the commitment of time, energy, and expertise that would ordinarily be associated with attaining such a high level of security.

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept. For example, if desired, the interlocking sub-units can each comprise a given time period of corresponding survival supplies. By one approach, a sub-unit may include sufficient supplies (such as food, hydration, and medical supplies) to sustain an individual for a seven day period, by interlocking four of these sub-units together a one month unit of supplies is created and by interlocking twelve sub-unit a twelve month unit is created.

I claim:

1. A method comprising:
accepting pre-catastrophe consideration-based private civil security subscriptions from subscribers with respect to providing civilly-catastrophic event-based access to survival supplies for authorized beneficiaries; and
creating physically interlocking sub-units of the survival supplies.

2. The method of claim 1 wherein creating physically interlocking sub-units of the survival supplies further comprises creating sub-units having an engagement mechanism to mate a plurality of the sub-units together.

3. The method of claim 2 wherein the engagement mechanism comprises at least one of:
a magnet;
a snap fit;
a clip;
a coupling structure;
a press-fit structure;
a hooks-and-loops fastener;
a hook;
a latch;
a threaded structure;
a clamp;
a formed joint;
a cable;
a keyed coupler.

4. The method of claim 1 wherein the sub-units are comprised of at least one of:
molded plastic;
corrugated plastic;
hard shell plastic;
Teflon;
corrugated cardboard;
wood;
steel;
carbon-based material;
aluminum;
composite material;
glass.

5. The method of claim 4 wherein the molded plastic is at least one of:
polypropylene;
polyethylene;
acrylontirile butadiene styrene;
polycarbonate;
polyvinyl chloride;
polyethylene tetraphthalate;
high impact polystyrene.

6. The method of claim 1 wherein the interlocking sub-units comprise at least one of:
a container;
a bin;
a box;
a case;
a drum;
a frame;
a chamber;
a bucket;
a basket;
a crate;
a trunk;
a carton;
a chest;
a coffer;
a barrel
a drawer,
a cabinet;
a tub;
a jar;
a pot;
a pan;
a bag.

7. The method of claim 2 wherein the sub-units comprise:
a base connecting four substantially vertical sides; and
a lid that can engage all four of the substantially vertical sides.

8. The method of claim 7 wherein an engagement between the lid and at least one of the substantially vertical sides is comprised of:
a hinge;
a magnet;
a snap fit;
a clip;
a press-fit structure;
a hooks-and-loops fastener;
a hook;
a latch;
a clamp;
a formed joint;
a bolt;
a cable;
a keyed coupler;
interlocking form.

9. The method of claim 8 wherein the lid and the at least one of the substantially vertical sides is configured and arranged to be at least one of:
selectively fixedly engaged; and
selectively removably engaged.

10. The method of claim 7 wherein the base of one of the sub-units is configured and arranged to selectively serve as the lid for another sub-unit.

11. The method of claim 7 wherein the lid further comprises at least one indentation for engaging a portion of another sub-unit.

12. The method of claim 11 wherein the base further comprises at least one projection for engaging a portion of another sub-unit.

13. The method of claim 12 wherein the indentation of the lid of one sub-unit mates with the projection of the base of another sub-unit such that the one sub-unit and the another sub-unit become at least substantially interlocked with one another.

14. The method of claim 7 wherein the lid further comprises at least one projection for engaging a portion of another sub-unit.

15. The method of claim 14 wherein the base further comprises at least one indentation for engaging a portion of another sub-unit.

16. The method of claim 15 wherein the indentation of the base of one sub-unit mates with the projection of the lid of another sub-unit such that the one sub-unit and the another sub-unit become at least substantially interlocked with one another.

17. The method of claim 7 wherein at least one substantially vertical side further comprises an indentation.

18. The method of claim 17 wherein at least one substantially vertical side further comprises a projection.

19. The method of claim 18 wherein the indentation of one sub-unit mates with the projection of another sub-unit such that the one sub-unit and the another sub-unit become at least substantially interlocked with one another.

20. The method of claim 7 wherein at least one of the sides, base, and lid further comprises, at least in part, a transparent member such that at least a portion of an interior area of the sub-unit can be viewed through the transparent member.

21. The method of claim 7 wherein at least one of the sides, base, and lid further comprises ventilation openings allowing for at least one of visibility and airflow.

22. The method of claim 7 wherein at least one of the sides, base, and lid further comprises ribbed reinforcements.

23. The method of claim 7 wherein at least one of the sides and lid further comprises a handle.

24. The method of claim 1 further comprising forming a unit of a plurality of the physically interlocking sub-units.

25. The method of claim 24 wherein forming the unit further comprises forming the unit by horizontally attaching sub-units that are disposed side-by-side.

26. The method of claim 24 wherein forming the unit further comprises forming the unit by vertically attaching sub-units that are disposed atop another.

27. The method of claim 24 wherein forming the unit further comprises forming the unit by horizontal attachment and vertical attachment of sub-units that are disposed side-by-side and atop one another.

28. The method of claim 24 wherein at least some of the plurality of sub-units as form the unit are sized and configured such that, once emptied of the survival supplies stored therein, are at least partially nestable within one another.

29. The method of claim 24 wherein the physically interlocking sub-units each contain a plurality of like resources.

30. The method of claim 1 wherein the plurality of physically interlocking sub-units comprise 18 sub-units, each dedicated to like resources.

31. The method of claim 1 further comprising compartmentalization of the sub-unit such that the survival supplies stored therein are segregated into compartments.

32. The method of claim 1 wherein the sub-units can be differentiated for identification using at least one of:
color;
a numbering system;
a lettering system;
shape;
size;
graphics;
symbols.
